(19) United States
(12) Patent Application Publication
(10) Pub. No.: US 2007/0224153 A1
(43) Pub. Date: Sep. 27, 2007

(54) High-deposition compositions and uses thereof

(76) Inventors: Joseph J. LiBrizzi, Hillsborough, NJ (US); Anthony J. Cossa, Branchburg, NJ (US); Russel M. Walters, Philadelphia, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/377,231

(22) Filed: Mar. 16, 2006

(51) Int. Cl. A61K 8/97; A61K 31/60; A61K 31/355; A61K 31/375; A61K 36/48

(52) U.S. Cl. 424/74; 424/757; 514/159; 514/458; 514/474; 514/725; 977/926

(57) ABSTRACT

Provided are compositions comprising actives associated with cationically-charged delivery systems, which compositions tend to exhibit relatively high-deposition of the actives onto the skin, nails, vagina and/or hair upon application thereto, and methods of use thereof.

### FIELD OF THE INVENTION

The present invention relates to compositions suitable for depositing actives on the skin or hair and, more particularly, to compositions comprising actives associated with cationically-charged delivery systems, which compositions tend to exhibit relatively high-deposition of the actives onto the skin, nails, vagina and/or hair upon application thereto.

### BACKGROUND

A wide variety of compositions comprising active agents intended for topical application to the skin and/or hair are known. For example, various conventional cleansing and other personal care compositions comprising vitamins, moisturizing agents, anti-UV agents, anti-inflammatory agents, and the like are commercially available.

Applicants have recognized, however, that many of such conventional compositions, including rinse-off and cream cleansers, tend to be relatively ineffective in depositing the actives associated therewith to the human body (i.e. the skin, hair, nails, vagina, etc.) in desirably high amounts. In particular, applicants have recognized that the use of conventional rinse-off products tends to result in most of the active agent present being washed away and relatively low amounts being left on the skin. While applicants do not wish to be bound by or to any theory of operation, it is believed by applicants that the surfactants present in such conventional products tend to emulsify the actives therein and prevent the adsorption of such actives to the human body. Applicants have also recognized that while many cream cleanser compositions tend to be more effective at depositing actives to skin than rinse-off cleansers, it is nevertheless desirable to achieve even more efficacious delivery of active agents to the skin for a variety of uses. Applicants note that conventional cream cleansers tend to be further disadvantageous in that they produce relatively low amounts of foam (often highly desirable in personal care compositions).

Other attempts to more effectively deposit actives on the skin using certain particles as delivery/carrier agents are described, for example, in U.S. Pat. No. 6,979,440 to Shefer et al. and U.S. Published Appl. No. 2005/0176598 to Bergquist et al. However, applicants have recognized that many surfactant effects on the efficacy of delivery (and other properties such as foaming) are unpredictable based on such disclosures and the art in general.

In light of the above, applicants have recognized the need for compositions that allow for the deposition of actives to the body in relatively high amounts. In certain embodiments, it is also desirable for such compositions to exhibit relatively high-foaming properties.

### SUMMARY

In one aspect of the present invention, provided are compositions comprising an active-delivery complex dispersed in a continuous phase, said composition being substantially free of anionic surfactants.

Another aspect of the present invention provides personal care products comprising a composition of the claimed invention.

In yet another aspect of the present invention, provided are methods of treating or preventing any of a variety of conditions of the skin, hair, nails, and/or vagina comprising contacting the skin with a composition of the present invention.

### DESCRIPTION OF PREFERRED EMBODIMENTS

Applicants have discovered unexpectedly that a wide variety of active agents may be associated with cationically-charged delivery systems and dispersed in a continuous agent to form compositions that overcome one or more of the disadvantages associated with conventional active-containing compositions, including conventional compositions using particulate delivery systems for delivering actives to the skin. That is, applicants have recognized that the compositions of the present invention, comprising an active-delivery complex dispersed in a continuous phase and being substantially free of anionic surfactants, tend to exhibit unexpectedly high active agent deposition and rinse-resistance properties as compared to conventional active-containing compositions and, in certain embodiments, tend to exhibit unexpectedly high-foaming characteristics.

In particular, applicants have measured the deposition and rinse resistance properties of compositions of the claimed invention using the Rinse Resistance Measurement described in detail below wherein the weight percentage of initial active remaining on the skin after rinsing with water for 15 seconds (% R₁₅) and 30 seconds (% R₃₀), based on the total weight amount of active originally applied to the skin, is measured and wherein, as will be recognized by those of skill in the art, a higher percentage (% R₁₅/% R₃₀) correlates to a desirably higher deposition and rinse resistance of the composition. Applicants have discovered that the present compositions tend to exhibit surprisingly high % R₁₅ and %R₃₀ values as compared to other comparable compositions. For example, in certain embodiments, the present compositions exhibit a % R₁₅ of about 12 or greater. In certain other preferred embodiments the present compositions exhibit a % R₁₅ of about 13 or greater, preferably about 14 or greater, more preferably about 15 or greater. In certain particularly preferred embodiments, the present compositions exhibit a % R₁₅ of about 20 or greater. Applicants have discovered that for certain preferred embodiments, such percentages tend to be at least about 1.1 times, more commonly from about 1.5 to as much as about 3 times or more greater than the % R₁₅ associated with comparable compositions outside of the scope of the present invention.

Furthermore, applicants have discovered unexpectedly that certain preferred compositions of the present invention also tend to exhibit relatively high-foaming properties, despite being substantially-free of anionic surfactants (which surfactants are known and used conventionally to increase foam levels associated with cleansing compositions). For example, applicants have measured the foam volume associated with certain preferred compositions of the present invention via the Foam Volume Test described hereinbelow. In certain embodiments, the present compositions exhibit a measured maximum foam volume (Fₘₐₓ) of about 200 or greater. In certain preferred embodiments the present compositions exhibit an Fₘₐₓ of about 250 or greater, preferably about 300 or greater, more preferably about 450 or greater, more preferably about 600 or greater, and more preferably about 700 or greater. Applicants have discovered that for certain preferred embodiments, such foam volumes tend to be at least about 1.5 times to as much as about 18 times or more greater than the volumes associated with relatively high active depositing cream cleanser compositions and tend to be comparable with the volumes of relatively low-depositing, but high foaming conventional compositions comprising significant amounts of anionic surfactant.

Any of a variety of active agents suitable for application to the skin, nails, vagina and/or hair may be used according to the present invention. Suitable active agents include ceramides, antioxidants, vitamins, moisturizing agents, anti-UV agents, keratolytic agents, anti-inflammatory agents, anti-aging agents, anti-bacterial agents, anti-dandruff agents, retinoids, pigments, fragrances, dyes, hydroxy acids, cooling agents, heating agents, anti-wrinkle agents, any additional actives listed in U.S. Patent Application Publication No. 20030053974 (incorporated herein by reference), combinations of two or more thereof, and the like. Certain preferred active agents include salicylic acid, retinol, vitamin E, vitamin C, jojoba oil, soybean, soybean extracts, combinations of two or more thereof, and the like. In certain particularly preferred embodiments, the active comprises salicylic acid.

Any particle, molecule, combinations of two or more thereof, and the like having a cationic charge associated therewith and being suitable for associating with, and facilitating delivery to the skin (or otherwise to the human body) of, an active agent may be used as a cationically-charged delivery system according to the present invention. Suitable cationically-charged delivery systems may comprise, for example, solid particles, polymers, polymer micelles, and polymer matricies, and the like.

In certain preferred embodiments, the cationically-charged delivery systems of the present invention comprise one or more particles. The particles may comprise one or more cationically-charged moieties adsorbed and/or incorporated therein. The particles may be of any suitable size, including for example, particles having a diameter of less than about 5000 nm, preferably from about 10 nm to about 5000 nm, more preferably from about 50 nm to about 2000 nm, more preferably from about 50 nm to about 1000 nm or from about 100 nm to about 2000 nm, and more preferably from about 100 nm to about 1000 nm. The particles may be made up of any suitable materials including certain preferred particles comprising waxes, such as synthetic waxes and/or natural waxes, polymers, copolymers, fats, and the like, as well as combinations of two or more thereof. Certain preferred particles include nanocapsules, nanoparticles, and nanospheres, such as those described, for example, in U.S. Pat. No. 6,979,440 (issued to Salvona, LLC) which is incorporated herein in its entirety, and the like. Particularly preferred cationically-charged particle suitable for use herein include cationically-charged solid nanospheres comprising synthetic wax and having a hydrophobic core.

As used herein, the term “active-delivery complex” refers to any complex formed by associating at least one active agent with at least one cationically-charged delivery system of the present invention. The active agent and cationically-charged delivery system may be associated to each other in any suitable manner to produce an active-delivery complex in accord with the present invention. For example, one of the active agent or delivery systems may be encapsulated or otherwise incorporated within the other, the active agent and delivery system may be chemically bound together via ionic, hydrogen, covalent, Vanderwaal, or other chemical bonding, combinations of two or more of such associations, and the like. In certain preferred embodiments, the active agent is incorporated or encapsulated in the delivery system. Preferably, the active-delivery complex of the present invention comprises a nanoparticle, nanocapsule, or nanosphere comprising an active incorporated or encapsulated therein. In certain more preferred embodiments, the active-delivery complex of the present invention comprises a solid nanosphere comprising a hydrophobic core containing an active agent.

Any suitable amounts of active may be used in the compositions of the present invention. In certain preferred embodiments, the compositions comprise from greater than zero to about 10 active weight percent of active agent, based on the total weight of the composition. In certain more preferred embodiments, the compositions comprise from about 0.01 to about 5 active weight percent, more preferably from about 0.1 to about 3 active weight percent, and even more preferably from about 1 to about 3 active weight percent of active agent. As used herein, unless otherwise specified, the term “active weight percent” of a material refers to the percent by weight of active amount of such material in a composition of the present invention, based on the total weight of the composition.

Any suitable amounts of active-delivery complex may be used in the compositions of the present invention. In certain preferred embodiments, the compositions comprise from greater than zero to about 25 active weight percent of complex. In certain more preferred embodiments, the compositions comprise from about 0.03 to about 15 active weight percent, more preferably from about 0.3 to about 12 active weight percent, and even more preferably from about 2 to about 10 active weight percent of active agent.

Any of a variety of suitable materials may be used as a continuous phase in accord with the present invention. According to preferred embodiments, the continuous phase is selected to be capable of dispersing the active-delivery complex therein, based at least in part on the size, phase, and polarity of the complex. As will be recognized by those of skill in the art, the continuous phase is preferably an aqueous continuous phase.

As used herein, the term “substantially-free of anionic surfactants” refers to a composition that comprises about 1 wt. % or less of total active anionic surfactants based on the total weight of the composition. Preferred compositions that are substantially-free of anionic surfactants are compositions comprising from about 0.5 wt. % or less, more preferably 0.1 wt. % or less, more preferably 0.01 wt. % or less, and more preferably 0.001 wt. % or less of total active anionic surfactants based on the total weight of the composition. Those of skill in the art will recognize that the terms “X % or less” used herein include, in certain preferred embodiments, amounts of from greater than zero percent to X % of anionic surfactant, as well as, in certain more preferred embodiments, zero percent of anionic surfactant. Examples of such anionic surfactants include alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl taurates, fatty alkyl sulfoacetates, alkyl phosphates, and mixtures of two or more thereof.

According to certain preferred embodiments, the compositions of the present invention further comprise one or more polymeric thickeners. Any of a variety of polymeric thickeners may be used in accord with the present invention. Thickeners may be classified as either naturally, or synthetically derived products. Examples of the former include starch, cellulose, alginate, and protein. These naturally occurring polymers incorporate building blocks of polysaccharide units, or amino acids, to provide efficient, water-soluble rheology modifiers. Specific examples of natural polymers include: hydroxyalkyl cellulose such as hydroxymethyl cellulose, ethylcellulose (EC), ethylhydroxy ethylcellulose (EHEC), hydroxylbutyl methylcellulose (HBMC), hydroxyethylcellulose (HEC), hydroxypropylcellulose (HPC), Methylcellulose (MC), hydroxypropyl methylcellulose (HPMC), hydroxyethyl ethylcellulose, hydroxyethyl methylcellulose (HEMC) Carboxymethylcellulose. Grafting of selected moieties onto the backbone of the more widely utilized natural products, such as starch and cellulose, provides for numerous modified versions of the products. Frequently, hydrophobic domains are grafted onto the aforementioned natural polymers. Specific examples of hydrophobically modified (hm) natural polymers include HMHEC, HMEC, HMEHEC etc. Specific examples of starch based polymeric thickener include: starch acetates (SAC), hydroxyethylstarch (HES), carboxymethylethylstarch. Additional thickeners include various natural gums such as guar gum, locust bean gum, karaya gum, and xanthan gum. Additionally suitable synthetic thickeners include acrylic-based polymers, of which there are three general classes. The first class is based on homopolymers of (meth)acrylic acid and copolymers of (meth)acrylic acid, (meth)acrylate esters, and maleic acid, among many others. This group is typically referred to as the alkali swellable (or soluble) emulsions (ASE). Modification of the structure of ASE polymers by addition of hydrophobic moieties defines the second class of synthetic rheology modifiers known as the hydrophobically modified, alkali swellable emulsions (HASE). The third class of synthetic rheology modifiers is the hydrophobically modified, ethoxylated urethane resins (HEUR). This group of polymers typically consists of polyethylene glycol units of varying length, connected by urethane linkages, and terminated with hydrophobic end groups. There are many commercially available polymeric thickeners that are appropriate for use: commercially available HMHEC include Natrosol Plus from Aqualon Co. (Wilmington, Del.); Quaternized HEC polymers such as the SoftCat SL series available from Amerchol Corp; and xanthan gum (available commercially as Keltrol CG-T from Kelco (Atlanta, Ga.)). Commercially available synthetic polymeric thickeners include: Carbopol, Aculyn and Acrosul. Certain preferred polymeric thickeners include natural gums, such as xanthan gum, and quarternized HEC polymers.

Any suitable amount of polymeric thickener may be used in the compositions of the present invention. Applicants have recognized that particularly stable dispersions of the present invention may be made via the use of sufficient amounts of thickener. In certain preferred embodiments, sufficient thickener is used to achieve compositions having a yield point (as measured via the Rheology measurement described herein below) of at least about 65 or greater. Preferably, sufficient thickener is used to achieve a yield point of about 85 or greater, more preferably of about 100 or greater, and in certain preferred embodiments, of about 120 or greater. In certain embodiments, the compositions of the present invention comprise from greater than zero to about 8 active weight percent of polymeric thickener, more preferably from about 0.1 to about 5, more preferably from about 0.2 to about 3, and more preferably from about 0.5 to about 1.5 weight percent of polymeric thickener.

According to certain embodiments, the compositions of the present invention may further comprise one or more nonionic, amphoteric, and/or cationic surfactants. Examples of suitable nonionic surfactants include, but are not limited to, fatty alcohol acid or amide ethoxylates, monoglyceride ethoxylates, sorbitan ester ethoxylates alkyl polyglycosides, mixtures thereof, and the like. Certain preferred nonionic surfactants include polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester. Examples of such preferred polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Delaware under the tradename, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Delaware under the tradename “Tween 20.” Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. Preferred alkyl gluocosides comprise from about 1 to about 6 glucose residues per molecule of alkyl glucoside. A preferred glucoside is decyl glucoside, which is the condensation product of decyl alcohol with a glucose polymer and is available commercially from Henkel Corporation of Hoboken, New Jersey under the tradename, “Plantaren 2000.”

As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions. Examples of amphoteric surfactants suitable for use in the present invention include, but are not limited to, amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof. Examples of suitable amphocarboxylate compounds include those of the formula:
A-CONH(CH₂)ₓN⁺R₅R₆R₇
wherein
A is an alkyl or alkenyl group having from about 7 to about 21, e.g. from about 10 to about 16 carbon atoms;
x is an integer of from about 2 to about 6;
R₅ is hydrogen or a carboxyalkyl group containing from about 2 to about 3 carbon atoms;
R₆ is a hydroxyalkyl group containing from about 2 to about 3 carbon atoms or is a group of the formula:
   R₈—O—(CH₂)ₙCO₂⁻
   wherein
   R₈ is an alkylene group having from about 2 to about 3 carbon atoms and n is 1 or 2; and
R₇ is a carboxyalkyl group containing from about 2 to about 3 carbon atoms;

Examples of suitable alkyl betaines include those compounds of the formula:
B—N⁺R₉R₁₀(CH₂)ₚCO₂⁻
wherein
   B is an alkyl or alkenyl group having from about 8 to about 22, e.g., from about 8 to about 16 carbon atoms;
   R₉ and R₁₀ are each independently an alkyl or hydroxyalkyl group having from about 1 to about 4 carbon atoms; and
   p is 1 or 2.
      A preferred betaine for use in the present invention is lauryl betaine, available commercially from Albright & Wilson, Ltd. of West Midlands, United Kingdom as “Empigen BB/J.” Examples of suitable amidoalkyl betaines include those compounds of the formula:
      D-CO—NH(CH₂)_{q}—N⁺R₁₁R₁₂(CH₂)ₘCO₂⁻
wherein
   D is an alkyl or alkenyl group having from about 7 to about 21, e.g. from about 7 to about 15 carbon atoms;
   R₁₁ and R₁₂ are each independently an alkyl or Hydroxyalkyl group having from about 1 to about 4 carbon atoms;
   q is an integer from about 2 to about 6; and m is 1 or 2.
      One amidoalkyl betaine is cocamidopropyl betaine, available commercially from Goldschmidt Chemical Corporation of Hopewell, Virginia under the tradename, “Tegobetaine L7.”

Classes of cationic surfactants that are suitable for use in this invention include alkyl quaternaries (mono, di, or tri), benzyl quaternaries, ester quaternaries, ethoxylated quaternaries, alkyl amines, and mixtures of two or more thereof, and the like.

Any suitable amounts of nonionic, amphoteric, and/or cationic surfactants may be used in the compositions of the present invention. According to certain preferred embodiments, the present compositions comprise about 0.3 active weight percent or greater of surfactants selected from the group consisting of nonionic, amphoteric, cationic surfactants, and combinations of two or more thereof. In certain more preferred embodiments, the compositions comprise about 0.7 or greater, more preferably about 1.5 active weight percent or greater, more preferably about 3 active weight percent or greater, more preferably about 7 active weight percent or greater and more preferably about 10 active weight percent or greater of surfactants selected from the group consisting of nonionic, amphoteric, cationic surfactants, and combinations of two or more thereof. Furthermore, in certain preferred embodiments, the present compositions comprise an amount of betaine surfactant of from about 0.1 to about 20 active weight percent, e.g. from about 0.5 to about 15 or from about 1.0 to about 10 active weight percent. In certain preferred embodiments, the present compositions comprise an amount of amphoteric surfactant that is not a betaine, of from about 0.1 to about 20 active weight percent, e.g. from about 0.5 to about 15 or from about 1.0 to about 10 active weight percent. In certain preferred embodiments, the present compositions comprise an amount of nonionic surfactant, preferably a glycoside, of from about 0.1 to about 20 active weight percent, e.g. from about 0.5 to about 15 or from about 1.0 to about 10 active weight percent. In certain preferred embodiments, the compositions comprise from about 0.3 to about 60, preferably from about 0.3 to about 45, more preferably from about 0.7 to about 20, more preferably from about 1 to about 20, and more preferably from about 3 to about 20 active weight percent of a combination comprising at least one betaine, at least one amphoteric surfactant that is not a betaine, and at least one nonionic surfactant, preferably a glycoside.

Any of a variety of commercially available pearlescent or opacifying agents are suitable for use in this invention. The pearlescent or opacifying agent may be present in an amount, based upon the total weight of the composition, of from about 1 percent to about 10 percent, e.g. from about 1.5 percent to about 7 percent or from about 2 percent to about 5 percent. Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO—(JO)ₐ—H, wherein J is an alkylene group having from about 2 to about 3 carbon atoms; and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the shampoo composition, and mixtures thereof

The pearlescent or opacifying agent may be introduced to the composition as a pre-formed, stabilized aqueous dispersion, such as that commercially available from Henkel Corporation of Hoboken, New Jersey under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and may be in a weight percent ratio of from about 25 to about 30: about 3 to about 15: about 20 to about 25, respectively.

Any of a variety of commercially available secondary conditioners are suitable for use in this invention. In one embodiment, the volatile silicone conditioning agent has an atmospheric pressure boiling point less than about 220° C. The volatile silicone conditioner may be present in an amount of from about 0 percent to about 3 percent, e.g. from about 0.25 percent to about 2.5 percent or from about 0.5 percent to about 1.0 percent, based on the overall weight of the composition. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Michigan under the tradename, “DC-345” and mixtures thereof, and preferably include cyclomethicone fluids.

Any of a variety of commercially available humectants, which are capable of providing moisturization and conditioning properties to the personal cleansing composition, are suitable for use in the present invention. The humectant may be present in an amount of from about 0 percent to about 10 percent, e.g. from about 0.5 percent to about 5 percent or from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, dipropylene glycol, and mixtures thereof; 2) polyalkylene glycol of the formula: HO—(R″O)_{b}—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH, wherein c is an integer from about 5 to about 25; 4) urea; and 5) mixtures thereof, with glycerine being the preferred humectant.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Michigan under the tradename, “Versene 100XL” and is present in an amount, based upon the total weight of the composition, from about 0 to about 0.5 percent or from about 0.05 percent to about 0.25 percent.

Suitable preservatives include Quaternium-15, available commercially as “Dowicil 200” from the Dow Chemical Corporation of Midland, Michigan, and are present in the composition in an amount, based upon the total weight of the composition, from about 0 to about 0.2 percent or from about 0.05 percent to about 0.10 percent.

Applicants have recognized that the compositions of the present invention may be used advantageously in a wide variety of applications. For example, in certain preferred embodiments, the present compositions are formulated to be, or be used in, personal care compositions and/or products such as, for example, anti-acne, anti-aging, moisturizers, sunscreens, make-up or make-removal compositions, conditioners, anti-itch, as well as other skin care, hair care, nail care, and women's health compositions, in particular cleansing compositions, and the like.

Applicants have recognized that the beneficial rinse-resistance and active delivery properties of the present compositions allow for efficient and effective treatment of acne, wrinkles, dermatitis, dryness, muscle pain, itch, and the like when applied to affected skin, hair, nails, or the like, and preventing the occurrence of such when applied to non-affected skin. Accordingly, the present invention provides methods of treating, mitigation, and/or preventing skin, vagina, hair, nail and skin-related conditions such as acne, wrinkles, dermatitis, dryness, muscle pain, itch, and the like, comprising the steps of contacting the skin with a composition, or product comprising a composition, of the present invention.

The compositions and/or personal care products of the present invention may be contacted with skin, hair, vagina and/or nails via any of a variety of means according to the present methods. For example, the compositions and products are preferably applied topically to the skin, hair, vagina and/or nails.

In certain preferred embodiments, the methods of the present invention further comprise the step of rinsing portion of the body having a composition of the present invention applied thereto to remove at least a portion of said composition therefrom. Any suitable conditions and suitable fluid for rinsing a composition from the skin, hair, vagina or nails may be used in the present inventions. In certain preferred embodiments, the rinsing step comprising rinsing with water, preferably from a tap. The rinsing step may further comprise applying pressure or rubbing the skin to rinse the composition applied thereto, and the like.

Any suitable amount of time may be allowed to pass in between the application and rinsing steps of the present methods. In certain preferred embodiments, the rinsing step is started less than 2 hours after the application step is completed. In certain more preferred embodiments, the rinsing step is started less than 1 hour, more preferably less than 30 minutes, more preferably less than 10 minutes, more preferably less than 1 minute, more preferably less than 30 seconds, and even more preferably about 15 seconds or less, after completion of the application step.

According to certain preferred embodiments, the methods of the present invention result in the deposition of relatively high amounts of actives to the skin. In certain embodiments, the methods of the present invention result in an weight percent of active on the skin after the rinsing step, based on the total weight originally deposited in the application step of at least about 12 or greater, preferably about 13 or greater, more preferably about 14 or greater, more preferably about 15 or greater, or more preferably about 20 or greater, as measured using the protocols as described below (or as appropriately and readily adapted for differing periods of time between application and rinsing.) The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

### Preparation of and Deposition Associated with Compositions Comprising Active-delivery Complexes

The cleansing compositions C1-C3, and C5 of the present invention and comparative compositions C4, C6, and C7 were prepared according to the materials and amounts listed in Table 1:

TABLE 1

| Trade Name | CTFA name | C1 | C2 | C3 | C4 | C5 | C6 | C7 |
|---|---|---|---|---|---|---|---|---|
| Purified Water | Purified Water | Qs | Qs | Qs | Qs | Qs | Qs | Qs |
| Keltrol CG-T | Xanthan Gum | — | 1.50 | — | — | 1.50 | 1.50 | 1.50 |
| SoftCAT ™ SL 100 | Polyquaternium-67 | — | — | 1.50 | — | — | — | — |
| Glycerin 917 (99%) | Glycerin | 1.00 | 1.00 | 1.00 | — | 1.00 | 1.00 | 1.00 |
| Tegobetaine L-7V (30%) | Cocamidopropyl Betaine | 12.00 | 12.00 | 12.00 | 25.00 | 12.00 | 12.00 | 12.00 |
| Monateric 949J (38%) | Disodium Lauroamphodiacetate | 2.00 | 2.00 | 2.00 | — | 2.00 | 2.00 | 2.00 |
| Plantaren 2000N (50%) | Decyl Glucoside | 6.00 | 6.00 | 6.00 | — | 6.00 | 6.00 | 6.00 |
| Rhodacal A 246L (40%) | Olefin Sulfonates (AOS) | — | — | — | 25.00 | 2.50 | 6.25 | 12.50 |
| Avanel S-150 | Sodium C12-15 | — | — | — | 8.00 | — | — | — |
| (35%) | Pareth-15 | | | | | | | |
| | Sulfonate | | | | | | | |
| Salvona Nanosal (20% | Sal Acid | 10.00 | 10.00 | 10.00 | 10.00 | 10.00 | 10.00 | 10.00 |
| Active Sal Acid) | | | | | | | | |
| NaoH 20% | pH adjuster | As | As | As | As | As | As | As |
| | | needed | needed | needed | needed | needed | needed | needed |

expressed as w/w %

The compositions of Table 1 were prepared as follows: water (50.0 parts) was added to a beaker. The polymer, if present, (Keltrol CG-T (CP Kelco, IL) in Example 2 and SoftCAT™ SL 100 (Dow, MI) in Example 3 was added to the water with mixing. The following ingredients were added thereto independently with mixing until each respective resulting mixture was homogenous: Tegobetaine L7V, Monateric 949J, Plantaren 2000N, Rhodacal A 246L and Glycerin 917. Then solid nanoparticles having salicylic acid incorporated therein (Savlona Nanosal) were added. The pH of the resulting solution was then adjusted with 20% Sodium Hydroxide solution until a final pH of about 4.2 to 4.4 was obtained. The remainder of the water was then added thereto.

The deposition and rinse-resistance associated with Compositions C1-C7 were measured via the Rinse Resistance measurement as described herein below and the results shown in Table 2. Two additional commercial formulas were tested and their results shown in Table 2. Composition C8, Oil Free Acne Wash (available commercially from Neutrogena, CA), is a conventional foaming facial cleanser that contains 2% salicylic acid and the following additional ingredients: Purified Water, Sodium C14-16 Olefin Sulfonate, Cocamidopropyl Betaine, Sodium C12-15 Pareth-15 Sulfonate, Aloe Barbadensis Leaf Extract, Anthemis Nobilis Flower Extract, Matricaria (Chamomilla Recutita) Flower Extract, Linoleamidopropyl PG-Dimonium Chloride Phosphate, Disodium EDTA, Propylene Glycol, FD&C Yellow 5, FD&C Red 40, Sodium Chloride, Fragrance. Composition C9, Deep Clean Cream Cleanser (available commercially from Neutrogena, CA), is a conventional cream cleanser that contains 2% salicylic acid and the following additional ingredients Water, Cetyl Alcohol, PPG-15 Stearyl Ether, Methyl Gluceth 20, Salicylic Acid, Steareth-21, Gelatin, Steareth-2, DEA-Cetyl Phosphate, Menthol, Polysorbate 60, Disodium EDTA, Fragrance.

TABLE 2

| | % | % |
|---|---|---|
| | Deposition | Deposition |
| | 15 s | 30 s |
| Formula | (% R₁₅) | (% R₃₀) |
| C1 | 14.6 | 10.7 |
| C2 | 21.5 | 5.7 |
| C3 | 13.9 | 9.8 |
| C4 | 7.7 | 6.4 |
| C5 | 12.7 | 2.3 |
| C6 | 7.6 | 2.8 |
| C7 | 7.4 | 1.3 |
| C8 | 7.6 | 4.9 |
| C9 | 11.6 | 8.7 |

Compositions 1, 2, 3, and 5 of the present invention have similar magnitudes of deposition that are unexpectedly high as compared to the comparative compositions. In particular, comparison of C1-3 and 5, each having about 1 active weight percent or less of anionic surfactant, to C4, C6, and C7 shows that the compositions of the present invention exhibit deposition/rinse-resistance that is at least about 1.6 to about 3 times compositions having greater than about 1 active weight percent of anionic surfactant.

Rinse Resistance Measurement Experimental Procedure:

The Rinse Resistance Measurements of the present invention are conducted via the following procedure:

Application and Rinsing

On the volar forearm of adult samples, 0.14 ml of test formula comprising active is applied to 23.7 cm² of skin. The formula is rubbed on the skin with an index finger for 30 s. The test area is then allowed to stand undisturbed for 15 s. After which, the test area is rinsed with tap water for 15 s. The water applied is sprayed through a PS2247 Sink Spay and Hose nozzle (PlumbShop, MI) from a line pressure of 41 psi. This nozzle produces a cone shaped spray pattern with angle of ˜7°. The test area on the skin is 24 cm from the nozzle. At this distance from the nozzle, the spray produced is approximately 3 cm in radius (approximately the same radius as the test area on the skin). After rinsing for the prescribed 15 s (approximately 1100 ml) the test area is measured as described below to determine the amount of active on the skin. Then the same test area is rinsed again in the same manner except for 30 s in duration, and them measured. Applicants note that the 15 s rinse time tends to represent typical consumer usage. Longer rinse times are used to observe differences between bases.

Measurement

If the active to be measured fluoresces with excitation (for example, salicylic acid, octyl methoxycinnamate (OMC), and 4-methylbenzylidene camphor (4-MBC), , and the like) then the following procedure using a SkinSkan® spectroflurometer (and not the HPLC method described below) is used to measure the amount of active in the test area for the Rinse Resistance Measurement. While the procedure below describes the actual measurements done for certain compositions comprising salicylic acid, those of skill in the art will be readily able to adapt the procedure to measure the rinse-resistance (% R15/% R₃₀) associated with any actives that fluoresce.

Salicylic acid areal densities on the skin were measured with a SkinSkan® spectroflurometer manufactured by Jobin Yvon Horiba (SPEX Industries, Edison, NJ). Excitation radiation from a 125 W xenon arc lamp was filtered through an excitation double monochromator (200±660 nm wavelength range and 1200 grooves per mm grating) and was focused onto one leg of a bifurcated quartz fiber optic bundle. The fiber optic bundle (2 mm total diameter) consisted of 62 fibers (214 mm in diameter each) and was used to deliver excitation radiation to the skin and emission radiation from the skin back to the spectrofluorimeter. Measurements were acquired by placing the fiber optic probe in contact with the skin. The incident excitation was 314 nm, and the emitted spectra collected from 330 to 540 nm. For each SkinSkan measurement 3 spectra were averaged together. The full test formula application, rinsing, and measurement procedure was conducted 3 times for each test formula.

To convert the raw fluorescence measurement to an areal density of salicylic acid, a calibration curve was created. Salicylic acid was dissolved in isopropanol (Fischer, NJ) to make solutions of 0.5, 1.0 and 2.0 wt %. With each of these solutions of known salicylic acid concentration, 0.14 ml was applied to a fresh 23.7 cm² area of volar form arm skin. The isopropanol was then allowed to evaporate and fluorescence was measured from this area on the skin in the same manner as described earlier. Additionally the fluorescence of native skin was measured. A calibration curve was then created with the flouresence intensity (a.u.) and the known salicylic acid areal density. The calibration curve was fitted with a power law as shown:
C=a(F−F₀)^{b}
where C is the areal density of salicylic acid, F is the fluorescence intensity of the test area, F₀ is the fluorescence intensity of native skin, and a and b are fitting parameters.

From the calibration curve the raw fluoresce intensity measured from the skin was converted to a salicylic acid areal density. The areal density was then converted to % deposited by dividing the areal density after each rinsing point by the total amount of salicylic acid applied from the formula before rinsing. The amount of salicylic acid initially applied was from a 0.14 ml of a formula containing 2% salicylic acid over a 23.7 cm² area, which is an areal density of 0.115 mg/cm².

If, and only if, the active to be measured in accord with the Rinse Resistance Measurement of the claimed invention does not fluoresce and is therefore not measurable using the a SkinSkan® spectroflurometer, then the following measurement procedure is substituted for the SkinSkan measurement described immediately above.

Tape Stripping/HPLC Experimental Method Description

Active areal densities on the skin can also be quantified with the following HPLC method. The active must first be removed or extracted from the skin, which can be accomplished by tape stripping.

Tape Stripping:

Square pieces (2.5 cm×2.5 cm) of adhesive tape (#5413, 3M, St. Paul, Minn.) are applied gently on the treated sites. The tapes are stripped after 1 min., and a total of 10 tapes are applied sequentially. The tapes are then placed into a 15 ml Falcon polypropylene tube (Becton Dickinson Labware, Franklin Lakes, N.J.). Methanol (3 ml in each tube, Burdick & Jackson, Muskegon, Mich.) is used to extract the active from the tapes. The solutions are sonicated for 30 min and stored in a refrigerator at <5° C. before HPLC analysis for quantification.

### Example 2

### Preparation and Comparison of Compositions Comprising Free Active with the Present Compositions

Compositions C10, comprising free salicylic acid not associated with a delivery system, and C11 of the present invention were prepared according to the materials and amounts listed in Table 3.:

TABLE 3

| Trade Name | CTFA name | C10 | C11 |
|---|---|---|---|
| Purified Water | Purified Water | Qs | Qs |
| Keltrol CG-T | Xanthan Gum | 1.50 | 1.50 |
| | Polysacchride | | |
| Glycerin 917 | Glycerin 917 | 1.00 | 1.00 |
| Tegobetaine L-7V (30%) | Cocamidopropyl | 24.00 | 24.00 |
| | Betaine | | |
| Monateric 949J (38%) | Disodium | 4.00 | 4.00 |
| | Lauroamphodiacetate | | |
| Plantaren 2000N (50%) | Decyl Glucoside | 12.00 | 12.00 |
| Salvona Nanosal | Sal Acid | — | 10.00 |
| (20% Active Sal Acid) | | | |
| Salicylic Acid | Salicylic Acid | 2.00 | — |
| NaoH 20% | pH adjuster | As needed | As needed |

The resulting deposition measurements for Compositions C10 and C11 are shown in Table 4 for each test formula and also for C2:

TABLE 4

| | % Deposition | % Deposition |
|---|---|---|
| | 15 s | 30 s |
| Formula | (% R₁₅) | (% R₃₀) |
| C10 | 5.4 | 2.1 |
| C2 | 21.5 | 5.7 |
| C11 | 15.7 | 2.9 |

As seen in Table 4, the deposition of C11 (encapsulated salicylic acid) is significantly higher (nearly 3 times greater) than the deposition of C10 (free salicylic acid). The encapsulation of the salicylic acid allows for much lower levels of surfactant and for greatly increased amounts of active deposition.

Applicants have observed the effect of increased surfactant level on the deposition of the two formulas (C2 and C11) with encapsulated salicylic acid. As can be seen in Table 4, with the surfactant level doubled from C2 to C11, the deposition is reduced, but only slightly. In systems with free salicylic acid, when the surfactant level is increased the deposition is strongly reduced.

### Example 3

### Foam Levels Associated with the Present Compositions

Foam Volume Test Experimental Procedure:

An industrially accepted means to measure the foam generation of the consumer product is the Sita Foam Tester R-2000 (SITA Messtechnik GmbH, Dresden Germany). Specifically designed to measure foam generation, the Sita Foam Tester consists of a jacketed sample vessel with and agitator. To represent the hard water of tap water, 0.36 g of calcium chloride is dissolved in 995 g of DI water. Five (5) grams of test formula is added to this solution and mixed until homogeneous. Then this 0.5% dilution of test formula is placed in the holding tank of the Sita Foam Tester. For each experimental run, 250 ml of solution is introduced into the test vessel and allowed to come to 30° C.±2° C. The agitator spins at 1300 rpm for 30 seconds, then the foam volume is measured. The agitation is repeated for a total of 9 cycles. The foam generation test is conducted 3 times for each test sample.

Compositions 1-3 tested for foam generation were prepared as was shown in Table 1. Compositions 8, 9, 12, and 13 are commercial formulas. Examples 8 and 12 are typical foaming cleansers, Neutrogena Oil-Free Acne Wash (Neutrogena Corp, CA) and Clean & Clear Continuous Control Body Wash (Johnson & Johnson, NJ), respectively, both of which contain 2% salicylic acid. Examples 9 and 13 are cream cleansers, Neutrogena Deep Clean Cream Cleanser (Neutrogena, CA) and Neutrogena Oil-Free Acne Wash Cream Cleanser (Neutrogena, CA), respectively, both of which contain 2% salicylic acid.

TABLE 5

| | | Foam Vol. |
|---|---|---|
| | Foam Vol. | (ml @ max) |
| Formula | (ml @ 90 s) | (Fₘₐₓ) |
| C1 | 168 ± 11 | 246 ± 29 |
| C2 | 363 ± 5 | 744 ± 3 |
| C3 | 328 ± 4 | 716 ± 11 |
| C8 | 583 ± 70 | 891 ± 14 |
| C12 | 483 ± 49 | 891 ± 12 |
| C9 | 25 ± 3 | 43 ± 3 |
| C13 | 119 ± 1 | 134 ± 10 |

As expected, the commercial foaming cleansers, Compositions 8 and 11, produced the largest foam volumes at both 90 s and also the maximum foam volume. While the creams cleansers, Compositions 9 and 13 generate significantly lower volumes of foam. Surprisingly, compositions 2 and 3 produce foam levels significantly higher than the cream cleansers (Compositions 9 and 13) for both the foam volume at 90 s and also the maximum foam volume. Compositions 2 and 3 also have significantly larger foam volumes than Composition 1. The addition of the polymers (Keltrol CGT in Example 2 and SL-100 in Example 3) resulted in significantly higher foam volumes than the comparative surfactant system of Composition 1.

### Example 4

### Preparation of Cleansing Compositions with Salicylic Acid and Rheology Associated therewith

The cleansing compositions 14 through 19 were prepared according to the materials and amounts listed in Table 6.:

TABLE 6

| Trade Name | CTFA name | C14 | C15 | C16 | C17 | C18 | C19 |
|---|---|---|---|---|---|---|---|
| Purified Water | Purified Water | Qs | Qs | Qs | Qs | Qs | Qs |
| Keltrol CG-T | Xanthan Gum | 1.00 | — | — | — | — | — |
| SoftCAT ™ SL 100 | Polyquaternium-67 | — | 0.25 | — | — | — | — |
| SoftCAT ™ SL 5 | Polyquaternium-67 | — | — | 0.70 | — | — | — |
| Polysurf 67 | Hydroxyethylcellulose | — | — | — | 1.00 | — | — |
| Natrosol 250 | Hydroxyethylcellulose | — | — | — | — | 1.00 | — |
| Amaze XT | Xanthan Gum | — | — | — | — | — | 1.00 |
| Glycerin 917 | Glycerin 917 | 1.00 | 1.00 | 1.00 | 1.00 | 1.00 | 1.00 |
| Tegobetaine L-7V | Cocamidopropyl Betaine | 12.00 | 12.00 | 12.00 | 12.00 | 12.00 | 12.00 |
| Monateric 949J | Disodium Lauroamphodiacetate | 2.00 | 2.00 | 2.00 | 2.00 | 2.00 | 2.00 |
| Plantaren 2000N | Decyl Glucoside | 6.00 | 6.00 | 6.00 | 6.00 | 6.00 | 6.00 |
| Rhodacal A 246L (40%) | Olefin Sulfonates (AOS) | — | — | 2.50 | 6.25 | 12.50 | 12.50 |
| Salvona Nanosal (20% | Sal Acid | 10.00 | 10.00 | 10.00 | 10.00 | 10.00 | 10.00 |
| Active Sal Acid) | | | | | | | |
| NaoH 20% | pH adjuster | As | As | As | As | As | As |
| | | needed | needed | needed | needed | needed | needed |

The compositions of Table 6 were prepared as follows: water (50.0 parts) was added to a beaker. The polymer, if present, (Keltrol CG-T (CP Kelco, IL) in Example 14 and SoftCAT™ SL 100 (Dow, MI) in Example 15, SoftCAT™ SL 100 (Dow, MI) in Example 16, Polysurf 67 (Hercules, Del.) in Example 17, Natrosol 250 (Hercuels, Del.) in Example 18, and Amaze™ XT (National Starch & Chemical, NJ) in Example 19) was added to the water with mixing. The following ingredients were added thereto independently with mixing until each respective resulting mixture was homogenous: Tegobetaine L7V, Monateric 949J, Plantaren 2000N, Rhodacal A 246L and Glycerin 917. Then the salicylic acid was added. The pH of the resulting solution was then adjusted with 20% Sodium Hydroxide solution until a final pH of about 4.2 to 4.4 was obtained. The remainder of the water was then added thereto.
Rheology Measurement (Oscillatory stress sweep)

Rheological measurements were conducted on a TA Instruments AR 2000 Rheometer (TA Instruments, New Castle, Del.). Cone and plate geometer with 1° and a diameter of 40 mm was used. The gap between the plates was set to 30 μm. All rheological measurements were preformed at 25° C., and a solvent trap was used to minimize evaporation during the experiment.

The oscillatory stress was increased from 0.10 Pa to 15920 Pa, while the frequency was held constant at 1.00 Hz. Ten (10) data points where collected over each decade of the oscillatory stress sweep. The yield point is the stress at which the linear-elastic range is exceeded. The yield point was defined in a manner consistent in the art and with, for example, the methodology described in Mezger, The Rheology Handbook, Vincentz Verlag (Hanover, Germany) 2002, pp. 33-36 and 134. That is, from a plot of the natural log (ln) of the stress, ln(stress), and the ln(strain). At low stress, there is a linear relationship between the ln(stress) and the ln(strain). At higher stress, near the yield point, the linear relationship breaks. To determine the yield point a linear relationship is fit to the data at low stress, and a second linear relationship is fit tangent to the region about the yield point. The yield point is defined as the intersection between the two linear equations. The yield points for Examples 1,2 and 14-19 are shown in Table 7 along with the 40° C. stability of the formula after 42 days.

TABLE 7

| | Yield Point | |
|---|---|---|
| | Pa | Stability |
| C1 | | Fail |
| C2 | 122.0 | Pass |
| C14 | 113.0 | Pass |
| C3 | 181.0 | Pass |
| C15 | 5.7 | Fail |
| C16 | 65.0 | Fail |
| C17 | 7.0 | Fail |
| C18 | 9.2 | Fail |
| C19 | 12.0 | Fail |

Only Examples 2, 14, and 3 were stable, as defined above. As can be seen in Table 7., the three stable Examples all have Yield Points greater than 113 Pa. Examples 15-19 were found to be unstable and all have yield points less then 66 Pa. The yield point required to provide a stable formula will vary with the specific constituents of the formula. For example larger encapsulation particles will likely require a larger yield value to ensure stability than smaller encapsulation particles.

As can be seen in Examples 3 and 15, by increasing the amount of polymeric thickener in the formula, in this case form 0.25% to 1.0% of Dow SL-100, a yield Point can be increased and the formula can be made to be stable.

We claim:

1. A method of treating a skin condition comprising applying to the skin a composition comprising an active-delivery complex dispersed in a continuous phase, said composition being substantially free of anionic surfactants.

2. The method of claim 1 wherein said skin condition is selected from the group consisting of acne, wrinkles, dermatitis, dryness, muscle pain, itch and combinations of two or more thereof.

3. The method of claim 2 wherein said composition has an % R₁₅ of about 13 or greater.

4. The method of claim 3 wherein said composition has an % R₁₅ of about 15 or greater.

5. The method of claim 1 wherein said active-delivery complex comprises one or more active agents selected from the group consisting of salicylic acid, retinol, vitamin E, vitamin C, jojoba oil, soybean, soybean extracts, and combinations of two or more thereof.

6. The method of claim 1 wherein said active-delivery complex comprises salicylic acid.

7. The method of claim 1 wherein said active-delivery complex comprises one or more cationically-charged solid particles.

8. The method of claim 1 wherein said active-delivery complex comprises a solid nanosphere comprising a hydrophobic core containing an active agent.

9. The method of claim 1 further comprising a polymeric thickener.

10. The method of claim 9 wherein said polymeric thickener is selected from the group consisting of natural gums and quarternized HEC polymers.

11. The method of claim 1 further comprising a surfactant selected from the group consisting of non-ionic surfactants, amphoteric surfactants, and combinations of two or more thereof.

12. The method of claim 11 comprising at least about 0.3 weight % of surfactant selected from the group consisting of non-ionic surfactants, amphoteric surfactants, and combinations of two or more thereof.

13. The method of claim 12 comprising at least about 1.5 weight % of surfactant selected from the group consisting of non-ionic surfactants, amphoteric surfactants, and combinations of two or more thereof.

14. The method of claim 13 comprising at least one amphoteric and at least one non-ionic surfactant.

15. The method of claim 14 wherein said at least one amphoteric comprises at least one betaine and at least one non-betaine amphoteric surfactant.

16. The method of claim 13 wherein said composition has an Fₘₐₓ of about 200 or greater.

17. The method of claim 13 wherein said composition has an Fₘₐₓ of about 700 or greater.

18. The method of claim 1 further comprising the step of rinsing at least a portion of the composition from the skin, nails, or vagina to which it is applied via the applying step.

19. A method of delivering an active to the human body comprising applying thereto a composition comprising a continuous phase; an active-delivery complex dispersed in said continuous phase; at least one surfactant selected from the group consisting of nonionic surfactants, amphoteric surfactants, and combinations of two or more thereof; at least one polymeric thickener; said composition being substantially free of anionic surfactants and exhibiting an % R₁₅ of about 12 or greater and an Fₘₐₓ of about 200 or greater.

20. The method of claim 19 wherein said active-delivery complex comprises a solid nanosphere comprising a hydrophobic core containing an active agent.

21. The method of claim 20 wherein said at least one surfactant comprises at least one betaine, at least one non-betaine amphoteric, and at least one non-ionic surfactant.

22. A method of delivering an active to the human body comprising applying thereto a composition comprising an active-delivery complex dispersed in a continuous phase and at least one amphoteric surfactant, said composition being substantially free of anionic surfactants and exhibiting an % R₁₅ of about 12 or greater.

23. The method of claim 22 wherein said active-delivery complex comprises a solid nanosphere comprising a hydrophobic core containing an active agent.

24. The method of claim 23 wherein said at least one amphoteric comprises at least one betaine and at least one non-betaine amphoteric surfactant.

25. The method of claim 24 further comprising at least one polymer thickener and at least one non-ionic surfactant.

26. A method of treating a condition of the skin, hair, nails, or vagina comprising delivering an active to the skin in accord with the method of claim 22.

27. The method of claim 1 wherein said condition is selected from the group consisting of dermatitis, dryness, itch, volume, odor, and combinations of two or more thereof.
